(19) United States
(12) Patent Application Publication
(10) Pub. No.: US 2016/0030676 A1
(43) Pub. Date: Feb. 4, 2016

(54) DEVICES FOR PREVENTION OF UNINTENTIONAL SYRINGE ASPIRATION

(71) Applicant: SMITHS MEDICAL ASD, INC., Rockland,, MA (US)

(72) Inventors: Timothy Bresina, Shoreview, MN (US); Michael Blomquist, Blaine, MN (US)

(21) Appl. No.: 14/782,040

(22) PCT Filed: Apr. 3, 2014
(86) PCT No.: PCT/US2014/032753

§ 371 Date: Oct. 2, 2015

Related U.S. Application Data
(60) Provisional application No. 61/808,082, filed on Apr. 3, 2013.
(60) Provisional application No. 61/813,402, filed on Apr. 18, 2013.
(60) Provisional application No. 61/815,122, filed on Apr. 23, 2013.

(51) Int. Cl. A61M 5/315; A61M 5/31; A61M 5/145

(52) U.S. Cl. CPC A61M 5/31501; A61M 5/1452; A61M 5/3146; A61M 5/31511; A61M 2205/3606

(57) ABSTRACT

A device for prevention of unintentional syringe aspiration includes a syringe cartridge. The syringe cartridge includes a syringe barrel having a first end and a second end, and an outlet at the second end. A plunger is slidably disposed in the barrel between the first end and the second end. A cap is engaged with the first end of the cartridge; and the cap includes a force element. When a substance in the barrel is cooled and frozen and then thawed, the force element tends to force the plunger to move back toward the second end of the barrel.

### TECHNICAL FIELD

This disclosure relates generally to syringes. More particularly, this disclosure relates to devices for prevention of unintentional syringe aspiration.

### BACKGROUND

Health care practitioners, and health care device and drug manufacturers, have been concerned with the proper handling, storage, and delivery to patients of substances for treatment of a variety of medical conditions. For example, in handling, storing, and delivering freeze-dried substances, rather complex and elaborate multi-compartment syringes and other devices have been utilized for reconstitution and delivery of lyophilized medicaments and the like. Some of these known devices have been, for example, directed to alleviating problems associated with gas bubble formations when water or other liquids are added to hygroscopic substances. However, the delivery to patients of substances in simply freezing cold or frozen states has also been problematic, particularly in syringe cartridges. For example, a gel-like or otherwise semi-liquid or flowable substance such as “levodopa-carbidopa intestinal gel” or “LCIG” (a compound for treatment of advanced Parkinson's disease) may be contained within a syringe cartridge in a freezing cold or frozen state until it is thawed for delivery from the cartridge to a patient via an intestinal catheter connected to an infusion pump into which the cartridge has been loaded. Initially, when the substance is cooled and frozen in a barrel of the syringe cartridge for handling and storage before use, expansion forces of the substance against a plunger in the barrel can cause an unintentional and undesirable displacement of the plunger backwardly in the cartridge. Then, when the substance in the cartridge is thawed for use in the pump before delivery to the patient, a vacuum may be created by the displaced plunger which, in turn, may unintentionally or undesirably pull air into (or “aspirate”) the syringe cartridge. Also, if the plunger does not move when the substance thaws in a particular syringe cartridge, then a vacuum may be created ahead of the plunger which, in turn, may unintentionally or undesirably pull air around the plunger and into (or “aspirate”) the syringe cartridge. Regardless of mode of action, such unintentional and undesirable aspiration of the cartridge could cause one or more problems as known to health care practitioners who are familiar with proper techniques in use of such infusion systems.

It would therefore be useful and advantageous to provide devices for prevention of unintentional syringe aspiration which may tend to prevent, to some useful degree, air from being unintentionally aspirated into syringes when medication therein is thawed; and which may also be relatively simple and inexpensive to produce.

### SUMMARY

This disclosure describes novel and inventive devices for prevention of unintentional syringe aspiration.

In an embodiment, a device for prevention of unintentional syringe aspiration includes a syringe cartridge. The syringe cartridge includes a syringe barrel having a first end and a second end, and an outlet at the second end. A plunger is slidably disposed in the barrel between the first end and the second end. A cap is engaged with the first end of the cartridge; and the cap includes a force element. When a substance in the barrel is cooled and frozen and then thawed, the force element tends to force the plunger to move back toward the second end of the barrel.

In another embodiment, a device for prevention of unintentional syringe aspiration includes a syringe cartridge. The syringe cartridge includes a syringe barrel having a first end and a second end, and an outlet at the second end. A plunger is slidably disposed in the barrel between the first end and the second end. A cap is engaged with the first end of the cartridge; and the cap includes a force element. When a substance in the barrel is cooled and frozen and then thawed, the force element tends to force the plunger to move back toward the second end of the barrel. The device for prevention of unintentional syringe aspiration is characterised in that air tends to be prevented from being unintentionally aspirated into the syringe, by action of the force element.

In another embodiment, a device for prevention of unintentional syringe aspiration includes a syringe cartridge. The syringe cartridge includes a syringe barrel having a first end and a second end, and an outlet at the second end. A plunger is slidably disposed in the barrel between the first end and the second end. A cap is engaged with the first end of the cartridge;

and the cap includes a force element. When a substance in the barrel is cooled and frozen and then thawed, the force element tends to force the plunger to move back toward the second end of the barrel. The force element is selected from a group consisting of a spring-like component, a foam-like component, and a combination of a spring-like component and a foam-like component.

In another embodiment, a device for prevention of unintentional syringe aspiration includes a syringe cartridge. The syringe cartridge includes a syringe barrel having a first end and a second end, and an outlet at the second end. A plunger is slidably disposed in the barrel between the first end and the second end. The plunger includes a force element. When a substance in the barrel is cooled and frozen the force element tends to compress, and when the substance in the barrel is then thawed the force element tends to decompress.

In another embodiment, a device for prevention of unintentional syringe aspiration includes a syringe cartridge. The syringe cartridge includes a syringe barrel having a first end and a second end, and an outlet at the second end. A plunger is slidably disposed in the barrel between the first end and the second end. The plunger includes a force element. When a substance in the barrel is cooled and frozen the force element tends to compress, and when the substance in the barrel is then thawed the force element tends to decompress. The device for prevention of unintentional syringe aspiration is characterised in that air tends to be prevented from being unintentionally aspirated into the syringe, by action of the force element.

In another embodiment, a device for prevention of unintentional syringe aspiration includes a syringe cartridge. The syringe cartridge includes a syringe barrel having a first end and a second end, and an outlet at the second end. A plunger is slidably disposed in the barrel between the first end and the second end. The plunger includes a force element. When a substance in the barrel is cooled and frozen the force element tends to compress, and when the substance in the barrel is then thawed the force element tends to decompress. The force element is selected from a group consisting of a spring-like component, a foam-like component, and a combination of a spring-like component and a foam-like component.

### BRIEF DESCRIPTION OF THE DRAWINGS

Devices for prevention of unintentional syringe aspiration are illustrated by way of example and not limitation in the figures of the accompanying drawings in which:

FIG. 1 is an illustration of an example of an infusion pump of the prior art.

FIG. 1A is an illustration of an example of a cartridge for the pump of FIG. 1.

FIG. 1B is an illustration of a component of the cartridge, taken along line 1B-1B in FIG. 1A.

FIG. 2 is an illustration of an example of a device for prevention of unintentional syringe aspiration.

FIG. 3 is an illustration of another example of a device for prevention of unintentional syringe aspiration.

FIG. 4 is an illustration of another example of a device for prevention of unintentional syringe aspiration.

### DETAILED DESCRIPTION

Devices for prevention of unintentional syringe aspiration, described in greater detail by way of examples herein, can effectively and advantageously provide means for returning syringe plungers to their pre-frozen locations within syringe barrels, and thereby aid in preventing unintentional aspiration. As will be described by example or otherwise herein, devices for prevention of unintentional syringe aspiration can be provided by way of force elements with syringe cartridge plungers, or by any suitable combinations of such elements.

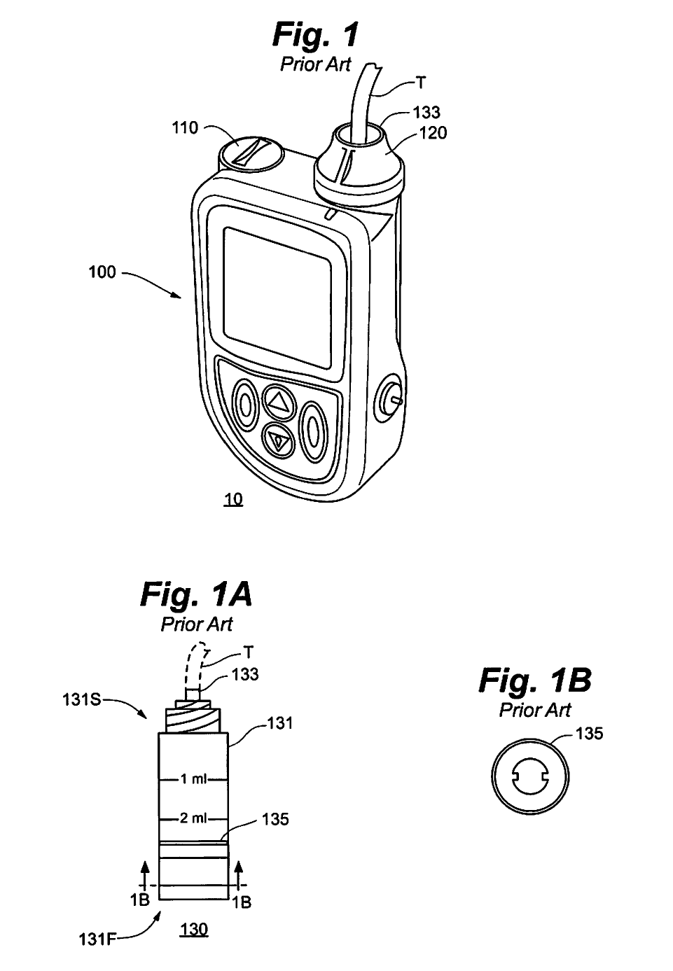

Referring to FIG. 1, therein illustrated is an example of a cartridge-based infusion pump of the prior art. Typically, such a known pump 10 includes a control unit 100 having a user interface comprising a display screen and input controls such as push-buttons and the like as are visible in the drawing. Pump 10 also includes a cap 110 on a battery compartment (not visible) and a cap 120 for a cartridge compartment (not visible). In use of pump 10, a syringe cartridge 130 as shown in FIGS. 1A and 1B contains a flowable substance. Cartridge 130 is inserted into the cartridge compartment of pump 10 with its outlet 133 oriented upwardly as in the drawings, and suitable tubing T as shown in FIG. 1 is connected to outlet 133 of cartridge 130 that is accessible through cap 120 of pump 10. As also shown in FIGS. 1A and 1B, syringe cartridge 130 includes a syringe barrel 131 having a first end 131F and a second end 131S where outlet 133 is located. A plunger 135 is slidably disposed in barrel 131 between first end 131F and second end 131S. Upon activation of pump 10, a driver mechanism (not visible) in pump 10 acts to drive or push (upwardly, in FIG. 1) against plunger 135 of cartridge 130 in the cartridge compartment of pump 10. Such sliding movement of plunger 135 in barrel 131 thereby causes the flowable substance to be forced outwardly from cartridge 130 at outlet 133. As understood by practitioners in the art, tubing T connected to outlet 133 serves as a conduit for the flowable substance from syringe cartridge 130 to a patient.

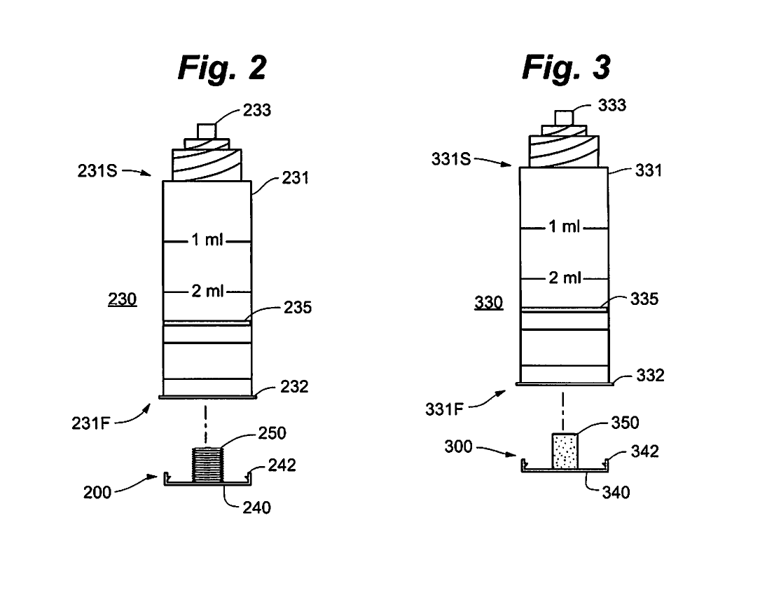

Referring to FIG. 2, therein illustrated is an example of a device for prevention of unintentional syringe aspiration 200 with a syringe cartridge 230. In this example, syringe cartridge 230 is capable of containing a flowable substance, and includes a substantially cylindrical syringe barrel 231 having a first end 231F and a second end 231S where an outlet 233 is located. Outlet 233 may include a removable cap or seal (not illustrated) to ensure that the substance remains within cartridge 230 until use; and such a cap or seal at outlet 233 may also inhibit contamination of the substance through outlet 233 before use. A plunger 235 is slidably disposed in barrel 231 between first end 231F and second end 231S. Device 200 includes a cap 240 which, as will be described, is removed before cartridge 230 is loaded into a pump. Cap 240 has a coupling portion 242 that is configured to engage complimentary structure of barrel 231 at first end 231F, to securely but removably hold cap 240 in position thereon. In the example of FIG. 2, coupling portion 242 is a circumferential, inward protrusion or ridge that is configured to removably snap into engagement or otherwise be mechanically and removably engaged with complimentary structure on barrel 231. As shown in the example of FIG. 2, this complimentary structure is a corresponding ridge or lip 232 at first end 231F of barrel 231. Cap 240 also includes a force element 250. In the example of FIG. 2, force element 250 comprises a spring-like component that is securely and centrally coupled to cap 240. In an embodiment of device 200, force element 250 could be a coil or another spring configuration of any suitable composition and construction.

In preparation and storage of syringe cartridge 230 before use, the flowable substance in barrel 231 can be cooled and frozen—as is typically done with substances such as LCIG as aforementioned. Upon cooling and freezing, such substances typically expand within barrel 231 between plunger 235 and end 231S. Consequently, plunger 235 can be unintentionally and undesirably displaced backwardly in barrel 231 toward end 231F. When cartridge 230 is then made ready for use with the pump, cartridge 230 and the substance therein is thawed for delivery to a patient. As thawing occurs and a volume occupied by the substance therefore decreases within barrel 231, a vacuum may then be created by plunger 235 as it is so displaced—which, in turn, may unintentionally or undesirably allow aspiration of syringe cartridge 230 as aforedescribed. However, when cap 240 is installed at end 231F of barrel 231 which contains the frozen substance, force element 250 advantageously tends to instead force plunger 235 to move back toward end 231S of barrel 231 as thawing occurs and the volume occupied by the previously frozen substance between plunger 235 and end 231S decreases. As force element 250 acts upon plunger 235 while the substance is thawing, there is therefore an advantageous tendency for plunger 235 to move back toward end 231S and thereby prevent unintentional aspiration of cartridge 230 that could otherwise occur. Before cartridge 230 is installed in the pump, cap 240 is removed from barrel 231. When cartridge 230 is installed in the pump, upon activation the driver mechanism within the pump drives or pushes against plunger 235 of cartridge 230 in the cartridge compartment, to cause sliding movement of plunger 235 toward end 231S and thereby cause the flowable substance to be forced outwardly from cartridge 230 at outlet 233.

Referring now to FIG. 3, therein illustrated is another example of a device for prevention of unintentional syringe aspiration 300 with a syringe cartridge 330. Similarly to the description of the example of FIG. 2, in this example syringe cartridge 330 is capable of containing a flowable substance, and includes a substantially cylindrical syringe barrel 331 having a first end 331F and a second end 331S where an outlet 333 is located. Outlet 333 may include a removable cap or seal (not illustrated). A plunger 335 is slidably disposed in barrel 331 between first end 331F and second end 331S. Device 300 includes a cap 340 which is removed before cartridge 330 is loaded into a pump. Cap 340 has a coupling portion 342 that is configured to engage complimentary structure of barrel 331 at first end 331F. In the example of

FIG. 3, this complimentary structure is a corresponding ridge or lip 332 at first end 331F of barrel 331. Cap 340 also includes a force element 350. In the example of FIG. 3, force element 350 comprises a foam-like component that is securely and centrally coupled to cap 340. In an embodiment of device 300, force element 350 could be a foam block or brick of any suitable composition, construction, and configuration.

In preparation and storage of syringe cartridge 330 before use, the flowable substance in barrel 331 can be cooled and frozen as aforementioned. When cap 340 is installed at end 331F of barrel 331 which contains the frozen substance, force element 350 advantageously tends to force plunger 335 to move back toward end 331S of barrel 331 as thawing occurs and the volume occupied by the previously frozen substance between plunger 335 and end 331S decreases. As force element 350 acts upon plunger 335 while the substance is thawing, there is therefore an advantageous tendency for plunger 335 to move back toward end 331S and thereby prevent unintentional aspiration of cartridge 330 that could otherwise occur. Before cartridge 330 is installed in the pump, cap 340 is removed from barrel 331.

Although the examples of FIGS. 2 and 3 have described force elements as being embodied in spring-like components and foam-like components, respectively, it is to be appreciated and understood that particular devices for prevention of unintentional syringe aspiration—as described by example or otherwise contemplated herein—could include any combinations of spring-like components and foam-like components, of any suitable compositions, constructions, and configurations.

It is also to be appreciated and understood that caps for syringes, such as are described by example or otherwise contemplated herein, could also serve a purpose of tending to prevent introduction of contaminates since the caps would cover the otherwise possibly open or exposed first ends of the syringes.

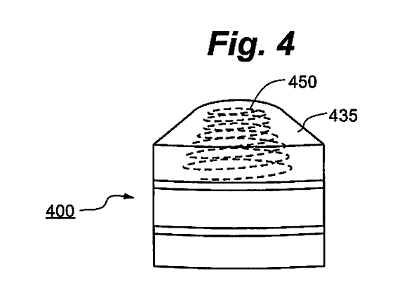

Referring now to FIG. 4, therein illustrated is another example of a device for prevention of unintentional syringe aspiration 400. In this example, a syringe cartridge (not illustrated) is capable of containing a flowable substance, and includes a substantially cylindrical syringe barrel having a first end and a second end where an outlet is located; and the outlet may include a removable cap or seal. A plunger 435 is slidably disposed in the barrel between the first end and the second end. Plunger 435 of device 400 also includes a force element 450. In the example of FIG. 4, force element 450 is provided internally, within plunger 435, and comprises a spring-like component (denoted schematically by phantom or dashed lines in the drawing). In a particular embodiment of device 400, force element 450 could be a coil or another spring configuration of any suitable composition and construction within plunger 435. In another embodiment, although not illustrated, force element 450 could comprise a foam-like component within plunger 435, such as a foam material of any suitable composition, construction, and configuration.

In preparation and storage of the syringe cartridge before use, the flowable substance in the syringe cartridge barrel can be cooled and frozen as aforementioned, with force element 450 in plunger 435 being thereby temporarily deformed or compressed toward the first end of the barrel by the freezing and expansion of the substance against it. Upon subsequent thawing, the volume occupied by the previously frozen substance in the barrel decreases, and plunger 435 and force element 450 advantageously decompress and expand back to their pre-frozen state toward the second end of the barrel. Plunger 435 and force element 450 thereby tend to prevent unintentional aspiration of the cartridge that could otherwise occur.

In another embodiment, and again although not illustrated, plunger 435 could itself have an inherently spring-like or foam-like property. Such characteristic could be provided by plunger material having, in whole or in part, a temporarily deformable or compressible capability. Such material would then return to a decompressed state when a force that tends to deform the material is diminished or removed (such as by the aforedescribed cycle of freezing and expansion, followed by thawing and decrease in volume of the substance in the cartridge). It is to be appreciated and understood that any suitable combinations could be utilized in such devices 400, of spring-like components, foam-like components, and materials having inherently spring-like or foam-like properties; and any suitable compositions, constructions, and configurations in various combinations could be provided for the devices as well.

Regardless of particular embodiments, it is to be appreciated and understood therefore that devices for prevention of unintentional syringe aspiration—as described by example or otherwise contemplated herein—effectively and advantageously provide means for returning syringe plungers to their pre-frozen locations within syringe barrels, and thereby aid in preventing unintentional aspiration. It is also to be appreciated and understood that devices for prevention of unintentional syringe aspiration that have been described by example, or which are otherwise contemplated herein, can be characterised in that they provide relatively simple and low-cost means for prevention of unintentional syringe aspiration.

It is also to be appreciated and understood that types, components, dimensions, fabrication processes, and other particulars and parameters of aforedescribed example embodiments can be substituted for others as desired, or that accessories can be added thereto. Thus, while devices for prevention of unintentional syringe aspiration have been particularly shown and described with reference to the accompanying figures and specification, it should be understood however that other modifications thereto are of course possible; and all of them are intended to be within the true spirit and scope of novel and inventive devices described herein. Accordingly, configurations and designs of various features could be modified or altered depending upon particular embodiments. For instance, the examples of the barrels and the novel and inventive caps as shown in FIGS. 2 and 3 could include materials that magnetically engage each other, with or without the complimentary coupling portions or structures as aforedescribed. As such, the barrels and caps could be magnetically coupled—or they could be both mechanically and magnetically coupled. It is also to be appreciated and understood that the aforementioned coupling portions and structures could be provided continuously, intermittently, or simply in selected regions of the barrels and the novel and inventive caps. Regardless of a particular embodiment, the coupling portions and structures are configured to function together satisfactorily, to securely but removably couple the novel and inventive caps to the barrels as aforedescribed.

Devices for prevention of unintentional syringe aspiration as described by example or otherwise contemplated herein could also include various combinations of the aforedescribed examples of force elements with various combinations of the aforedescribed mechanical and magnetic coupling portions and structures. Thus, for example, in situations where there is a high likelihood of unintentional aspiration, a combination of all of these features might be advantageously employed.

Regardless of particular components or modes of action, it is to be appreciated and understood that devices for prevention of unintentional syringe aspiration—such as have been described by example or are otherwise contemplated herein—can provide relatively simple and low-cost means for prevention of unintentional syringe aspiration. It is also to be appreciated and understood that devices for prevention of unintentional syringe aspiration, as have been described by example or otherwise contemplated herein, could potentially be used for or with virtually any devices which utilize syringe cartridges or similar components.

It is further to be understood that dimensioning and scaling of the drawings herein have been chosen to clearly show details of example embodiments. Thus, in some embodiments it is possible that spacing between, or orientations of, various features might be variable and visually different from those illustrated. Furthermore, although illustrations herein have depicted them as being generally cylindrical, it is to be understood that devices for prevention of unintentional syringe aspiration—and syringes cartridges utilizing them—may be provided in any complimentary or compatible dimensions or geometries. In any event, dimensioning and scaling could vary significantly across various embodiments thereof.

It is additionally to be understood in general that any suitable alternatives may be employed to provide novel and inventive devices for prevention of unintentional syringe aspiration such as those that are described by example or otherwise contemplated herein.

Lastly, compositions, sizes, and strengths of various aforementioned components of devices for prevention of unintentional syringe aspiration that are described by example or otherwise contemplated herein are all a matter of design choice depending upon intended uses thereof.

Accordingly, these and other various changes or modifications in form and detail may also be made, without departing from the true spirit and scope of devices for prevention of unintentional syringe aspiration that may be defined by the appended claims.

1. A device for prevention of unintentional syringe aspiration, comprising:
a syringe cartridge including a syringe barrel having a first end and a second end, and an outlet at the second end;
a plunger that is slidably disposed in the barrel between the first end and the second end; and
a cap engaged with the first end of the cartridge, the cap including a force element,
wherein when a substance in the barrel is cooled and frozen and then thawed, the force element tends to force the plunger to move back toward the second end of the barrel.

2. The device for prevention of unintentional syringe aspiration of claim 1, characterized in that air tends to be prevented from being unintentionally aspirated into the syringe, by action of the force element.

3. The device for prevention of unintentional syringe aspiration of claim 1, wherein the force element is selected from a group consisting of a spring-like component, a foam-like component, and a combination of a spring-like component and a foam-like component.

4. A device for prevention of unintentional syringe aspiration, comprising:
a syringe cartridge including a syringe barrel having a first end and a second end, and an outlet at the second end; and
a plunger that is slidably disposed in the barrel between the first end and the second end, the plunger including a force element,
wherein when a substance in the barrel is cooled and frozen the force element tends to compress, and when the substance in the barrel is then thawed the force element tends to decompress.

5. The device for prevention of unintentional syringe aspiration of claim 4, characterized in that air tends to be prevented from being unintentionally aspirated into the syringe, by action of the force element.

6. The device for prevention of unintentional syringe aspiration of claim 4, wherein the force element is selected from a group consisting of a spring-like component, foam-like component, and a combination of a spring-like component and a foam-like component.
